(19) United States
(12) Patent Application Publication
(10) Pub. No.: US 2008/0140102 A1
(43) Pub. Date: Jun. 12, 2008

(54) Medical instrument for removing objects from tight passages

(76) Inventors: Stuart I. Brown, St. Andrews (GB); Timothy Graham Frank, Wormit Newport-On-Tay (GB); Leslie Kelly, Cupar (GB); Rodney Mountain, Bruton (GB); Ian Rutherford, GB Dundee (GB)

Correspondence Address: ST. ONGE STEWARD JOHNSTON & REENS, LLC, 986 BEDFORD STREET, STAMFORD, CT, 06905-5619, US

(21) Appl. No.: 11/950,919

(22) Filed: Dec. 5, 2007

(30) Foreign Application Priority Data
Dec. 8, 2006 (DE) 10 2006 057 900.3

(51) Int. Cl. A61F 11/00

(52) U.S. Cl. 606/162

(57) ABSTRACT

The invention relates to a medical instrument for removing objects from narrow passages, having a shaft and an actuation element that is mounted in the shaft and on whose distal end a hook-shaped angled piece can be configured so that the distal end of the actuation element can be converted by a handle between an essentially extended form and a form that comprises the hook-shaped angled piece. It is proposed with the invention that the handle should be configured as consisting of at least two gripping members configured at an angle with respect to the longitudinal axis of the instrument, so that at least one gripping member can rotate with respect to the at least one other gripping member.

### CROSS-REFERENCE TO RELATED APPLICATIONS

The present application claims priority of German patent application No. 10 2006 057 900.3 filed on Dec. 8, 2006, the content of which is incorporated herein by reference.

### FIELD OF THE INVENTION

The invention relates to a medical instrument for removing objects from tight passages, having a shaft and, mounted in the shaft, an actuation element on whose distal end a hook-shaped angled piece can be configured, so that the distal end of the actuation element can be displaced by a handle between an essentially extended form and a form that comprises the hook-shaped angled piece.

### BACKGROUND OF THE INVENTION

Hook-shaped medical instruments of this kind are known in the art, for instance from the ear, nose, and throat specialty, for removing cerumen, so-called earwax, or other alien material from the outer auditory canal. The disadvantage with known instruments is that it is difficult to manage to place the hook-shaped distal end of the shaft behind the object that is to be removed in order to extract the object then out of the auditory canal by means of the hook. Depending on the particular special circumstances, there is always the danger in the art that on inserting the hook-shaped shaft, the object to be removed can be pressed farther into the auditory canal.

A medical instrument of this type is known for instance from patent DE 195 14 534 C2. In this known medical instrument the handle is configured as a rod-shaped extension of the shaft. This rod-shaped handle, essentially round in cross-section, makes possible a slender and compact structure for the instrument, but the rod-shaped configuration does not ensure any constantly secure and immovable positioning in the user's hand.

It is consequently the object of the invention to produce a medical instrument for removing objects from narrow passages that ensures simple and secure handling along with reliable removal of objects even from extremely tight spaces.

### SUMMARY OF THE INVENTION

The invention fulfills this object in a manner marked by the fact that the handle is configured as consisting of at least two gripping members in such a way that the handle consists of at least two gripping members configured at an angle to the instrument's longitudinal axis, and at least one gripping member can rotate with respect to the at least one other gripping member.

Owing to the invention's design of the handle as a grip having a scissors-type configuration and set off at an angle to the instrument's longitudinal axis, consisting of at least two gripping members, the medical instrument so configured by this invention sits securely and non-slippably in the user's hand, thus allowing a simple, precise, and secure handling of the instrument.

According to a practical embodiment of the invention it is proposed that the handle should comprise a fixed gripping member connected with the shaft and also a gripping member that can rotate with respect to the fixed gripping member, so that the rotatable gripping member is in active connection with the actuation element. Use of the rotatable gripping member that is in active connection with the actuation element makes possible a measured axial sliding of the actuation element and thus a treatment that is secure and does not endanger the patient.

According to the invention, the distal end of the actuation element is advantageously converted into the hook-shaped form set at an angle by self-actuation, which is made possible through the use of an actuation element consisting of pre-bent material.

Appropriate materials for inventive configuration of at least the distal end of the actuation element include, in particular, so-called super-elastic materials such as NiTi alloys, which possess the necessary elasticity and resilience to be bent into an essentially extended form on the one hand, and on the other hand to reassume the pre-bent hook shape thanks to the memory effect, and so that they are sufficiently stable in the angled form to be able to withdraw from the passage the object that is to be removed, even when confronted with a certain resistance.

To configure the distal end of the actuation element it is proposed, according to a first embodiment of the invention, that the distal end of the actuation elements should be configured as a closed wire loop.

With a second embodiment of the invention it is proposed that the distal end of the actuation element should be configured as a striated flat sheet metal.

It is further proposed with the invention that the free distal end of the actuation element should have a blunted configuration to rule out the causing of any damage to the passage that is to be examined, such as by the free end of the actuation element that is first to be inserted into the passage in the extended form.

Finally it is proposed with the invention that the shaft should be capable of insertion into the passage to be examined by means of a speculum. These funnel-shaped instruments are known for instance from the ear, nose, and throat specialty and used for insertion into body cavities such as the outer auditory canal. The speculum prevents damage to be caused to the passage that is to be examined owing to the insertion of diagnostic instruments.

Further characteristics and advantages of the invention can be seen from the appended illustrations, in which two embodiments of the inventive medical instrument for removing objects from narrow passages are depicted in schematic fashion, without restricting the invention to these illustrated embodiments.

### BRIEF DESCRIPTION OF THE DRAWINGS

FIG. 1 shows a schematic side view of an inventive medical instrument with the shaft included.

FIG. 2 shows a perspective view of detail II from FIG. 1, depicting a first embodiment of the distal end of the actuation element.

FIG. 3 shows a perspective view of detail II from FIG. 1, depicting a second embodiment of the distal end of the actuation element.

FIG. 4a shows a schematic view of the use of an inventive medical instrument during insertion.

FIG. 4b shows a view according to FIG. 4a but depicting the working position.

FIG. 5 shows a schematic side view of a speculum.

### DETAILED DESCRIPTION OF THE INVENTION

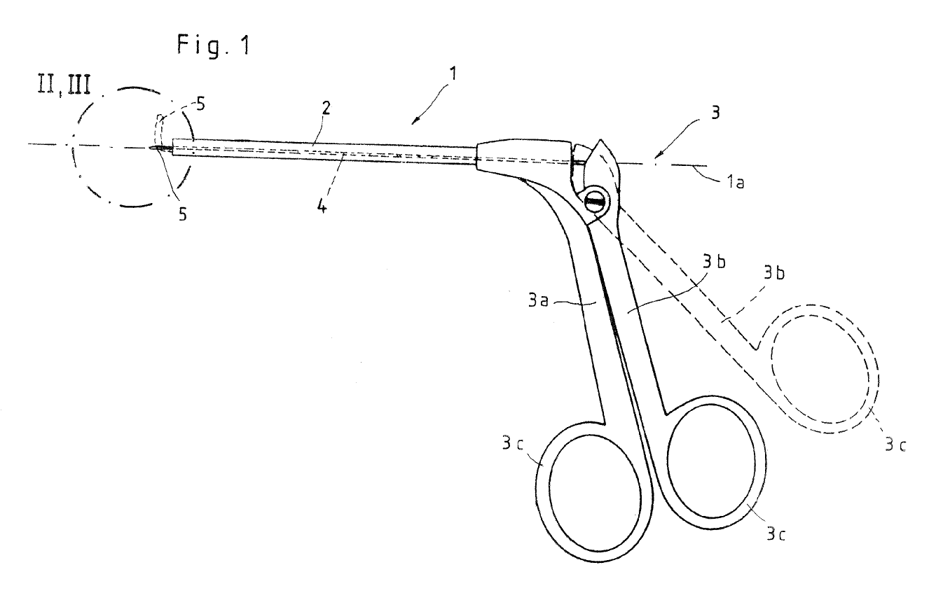

The medical instrument for removing objects from narrow passages, illustrated schematically and only as an example in FIG. 1, consists essentially of a shaft 2 on whose proximal end a handle 3 is configured that consists of a fixed gripping member 3a connected with the shaft and of a gripping member 3b that can rotate with respect to the fixed gripping member 3a. To facilitate holding and operating of the handle 3, both gripping members 3a and 3b in addition comprise ringer rings 3c.

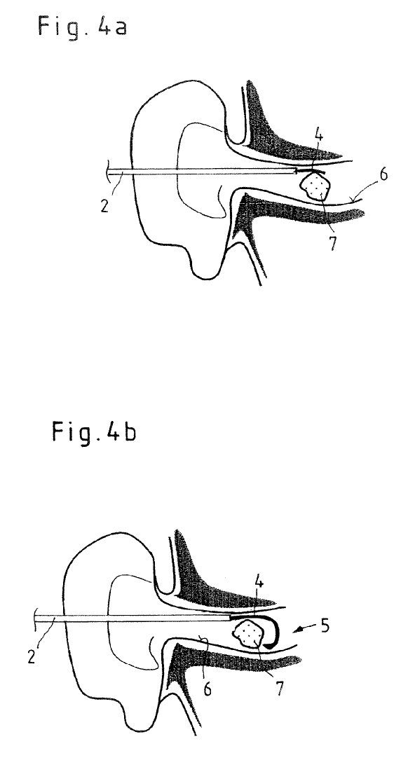

Positioned inside the shaft 2 is an actuation element 4 that is in active connection with the rotatable gripping member 3b of the handle 3 and whose distal end, forming an angled piece 5, can be slid out of the distal end of the shaft 2 in order to be able to grasp a foreign body 7 positioned in a passage 6 from behind, as shown in particular in FIG. 4b.

As can be seen from FIG. 1, the angled piece 5, configured on the distal end of the actuation element 4, and the rotatable gripping member 3b of the handle 3 are in active connection with one another by means of the actuation element 4 mounted in the shaft 2, in such a manner that, through the displacement of the gripping member 3b of the handle 3, the distal end of the actuation element 4 can be converted from the starting position (filled-in depiction in FIG. 1) into the working position (dotted depiction in FIG. 1), moved out of the shaft 2 and forming the angled piece 5, or vice versa.

The respective corresponding position of the rotatable gripping member 3b of the handle 3 is likewise illustrated filled in FIG. 1 (for the extended starting position) and dotted (for the angled working position).

To avoid injury to the passage 6, for instance to the external auditory canal of an ear, and to facilitate insertion of the shaft 2 into the auditory canal 3, in practice a funnel-shaped speculum 8 is first inserted into the auditory canal 6. The speculum 8 shown in FIG. 5 was omitted from FIGS. 4a and 4b in order to make the major spatial relationships clearer.

To configure the hook-shaped angled piece 5 on the distal end of the actuation element 4, the distal end of the actuation element 4, at least, consists of an elastic material, such as a super-elastic NiTi alloy, that is pre-bent in a hooked shape. The elasticity of the material, however, allows the shaft to be converted into an essentially extended form in order to be able to insert it into the passage 6 that is to be examined by means of the shaft 2. As soon as the distal end of the actuation element 4 has again moved out of the shaft 2, it resumes the pre-bent hooked shape by self-actuation.

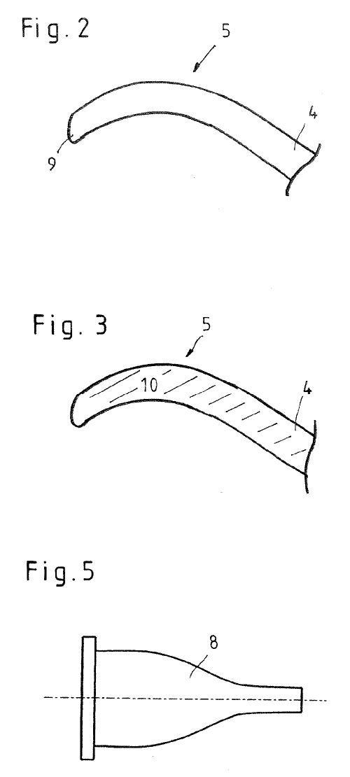

FIGS. 2 and 3 schematically present two embodiments for configuring the distal end of the actuation element 4, which forms the angled piece 5.

In the first embodiment, shown in FIG. 2, the distal end of the actuation element 4, which forms the angled piece 5, is configured as a closed wire loop 9. On the other hand, the second embodiment, shown in FIG. 3, shows the configuration of the distal end of the actuation element 4, which forms the angled piece 5, as a striated flat sheet metal 10.

As can further be seen from FIGS. 1 and 2, it is possible to configure the angled piece 5 in such a way that it bends at an angle away from the instrument's longitudinal axis 1a upward or downward. It is also possible, of course, to configure a lateral deflection of the angled piece 5.

The handling of the medical instrument 1 described so far for removing objects 7 from narrow passages operates as follows:

FIGS. 4a and 4b are schematic depictions of the practical use of the described medical instrument showing the example of a foreign body 7 that is to be removed from the outer auditory canal 6 of an ear, for instance a body such as hardened cerumen or earwax.

The speculum 8 shown in FIG. 5 was omitted from FIGS. 4a and 4b in the interests of showing a clearer general view. Likewise the illustration does not show other medical instruments such as an otoscope for visual monitoring of treatment.

The shaft 2 of the medical instrument 1 is inserted into the auditory canal 6 using the speculum 8 until the distal end of the shaft 2 has almost reached the foreign body 7 that is to be removed. Then the user of the medical instrument 1 rotates the rotatable gripping member 3b of the handle 3 into the position represented in dotted coloring in FIG. 1, so that the distal end of the actuation element 4 is pushed out of the distal end of the shaft 2, as shown in FIG. 4a.

The distal end of the actuation element 4 that is emerging from the shaft 2 passes the foreign body 7 that is to be removed and, upon further pushing outward of the actuation element 4 from the shaft 2, self-actuates itself to configure the pre-formed angled piece 5 behind the foreign body 7 because of the pre-tensing and the memory effect of the material; this can be seen from FIG. 4b.

To remove the foreign body 7 from the auditory canal 6, the user of the medical instrument 1 now needs only to withdraw the shaft 2 outward out of the auditory canal 6, with the gripping member 3b further rotated. The angled piece 5 that extends behind the foreign body 7 takes it along and removes it from the auditory canal 6.

In addition to the selection of an appropriately elastic material for configuring the distal end of the actuation element 4 in order to allow a repeated stretching and bending back of the distal end of the actuation element 4, the material should be selected with a view toward material resiliency and/or material strength in such a way that the angled piece 5 that grips behind the foreign body 7 that is to be removed does not immediately bend back when a resistance force is exerted on the hook-shaped angled piece 5 by the foreign body 7.

A medical instrument of this type of configuration for removing objects 7 from tight passages 6 is distinguished in that it combines simple structure and ease of operation with a guarantee of reliably removing foreign bodies 7 from the passage 6 to be examined. Thanks to the configuration of the handle 3 as a scissors-shaped grip having at least two gripping members 3a, 3b, the medical instrument 1 configured in this way is held securely and firmly in the user's hand and thus allows for a simple, precise, and secure handling of the medical instrument 1.

1. A medical instrument for removing objects from narrow passages, having a shaft and an actuation element that is mounted in the shaft and on whose distal end a hook-shaped angled piece can be configured so that the distal end of the actuation element can be converted by a handle between an essentially extended form and a form that comprises the hook-shaped angled piece, characterized in that the handle consists of at least two gripping members configured at an angle with respect to the instrument's longitudinal axis, so that at least one gripping member can rotate with respect to the at least one other gripping member.

2. A medical instrument according to claim 1, characterized in that the handle comprises a fixed gripping member connected with the shaft and a gripping member that can rotate with respect to the fixed gripping member so that the rotatable gripping member is in active connection with the actuation element.

3. A medical instrument according to claim 1, characterized in that the distal end of the actuation element actuates itself to assume the form that comprises a hook-shaped angled piece.

4. A medical instrument according to claim 1, characterized in that the distal end of the actuation element consists of a pre-bent elastic material.

5. A medical instrument according to claim 4, characterized in that the distal end of the actuation element consists of an NiTi alloy.

6. A medical instrument according to claim 1, characterized in that the distal end of the actuation element is configured as a closed wire loop.

7. A medical instrument according to claim 1, characterized in that the distal end of the actuation element is of striated configuration.

8. A medical instrument according to claim 1, characterized in that the free distal end of the actuation element is of blunted configuration.

9. A medical instrument according to claim 1, characterized in that the shaft can be inserted into the passage by a speculum.

10. A medical instrument according to claim 2, characterized in that the distal end of the actuation element actuates itself to assume the form that comprises a hook-shaped angled piece.
